(19) United States
(12) Patent Application Publication
(10) Pub. No.: US 2020/0362877 A1
(43) Pub. Date: Nov. 19, 2020

(54) RESPIRATION THERAPY APPLIANCE, AND FAN IMPELLER FOR A RESPIRATION THERAPY APPLIANCE

(71) Applicant: Loewenstein Medical Technology S.A., Luxembourg (LU)

(72) Inventor: Angela GERLACH, Hamburg (DE)

(21) Appl. No.: 15/931,666

(22) Filed: May 14, 2020

(30) Foreign Application Priority Data
May 16, 2019 (DE) 102019112864.1

(51) Int. Cl. F04D 29/32; F04D 29/38; F04D 19/00; A61M 16/00

(52) U.S. Cl. CPC F04D 29/325; F04D 29/384; F05D 2240/307; A61M 16/00; F04D 19/002

(57) ABSTRACT

The present invention relates to a respiration therapy appliance which comprises a fan for generating a respiratory air flow for carrying out respiration therapy. The fan comprises at least one rotatable fan impeller having a plurality of blade elements. At least some of the blade elements are equipped with in each case at least one winglet running at least in part on at least one axial longitudinal side of the blade element.

### CROSS-REFERENCE TO RELATED APPLICATIONS

The present application claims priority under 35 U.S.C. § 119 of German Patent Application No. 102019112864.1, filed May 16, 2019, the entire disclosure of which is expressly incorporated by reference herein.

### BACKGROUND OF THE INVENTION

### 1. Field of the Invention

The present invention relates to a respiration therapy appliance having at least one fan for generating a respiratory air flow for carrying out respiration therapy. The fan comprises at least one rotatable fan impeller having a plurality of blade elements.

### 2. Discussion of Background Information

Such appliances are used, for example, for ventilation or respiratory assistance or for cough assistance. To ensure that the treatment or therapy does not cause annoyance, the fan should make as little noise as possible during operation. For targeted respiration therapy, it is also important that adjustments to the speed of rotation of the fan impeller should take place as smoothly and as quickly as possible.

In view of the foregoing, it is desirable to have available a respiration therapy appliance that is able to advantageously meet the aforementioned requirements.

### SUMMARY OF THE INVENTION

The present invention provides a respiration therapy appliance and with a fan impeller set forth in the independent claims. Developments and advantageous embodiments are the subject matter of the dependent claims. Further advantages and features will become apparent from the general description and from the description of the illustrative embodiments.

The respiration therapy appliance according to the invention comprises at least one fan for generating a respiratory air flow for carrying out respiration therapy. The fan comprises at least one rotatable fan impeller. The fan impeller comprises a plurality of blade elements. At least some of the blade elements are equipped with in each case at least one winglet. The winglet runs at least in part on at least one axial longitudinal side of the blade element.

The respiration therapy appliance according to the invention may afford many advantages. One considerable advantage may be afforded by the blade elements equipped with the winglets. The fan impeller is thereby suitable and designed to particularly effectively and reliably suppress the exchange flow from the pressure side to the suction side of the blade elements and the exchange flow from the side space to the blade channels. A particular advantage of the invention may be that the suppression of the exchange flow is achieved with a particularly low weight of the component parts and moreover with a simple design. Thus, the fan impeller of the invention is considerably lighter than a fan impeller with a cover disk or other disks for reducing exchange flows. In this way, the fan impeller has a particularly low inertia, such that the speed of rotation of the fan impeller can be adjusted particularly smoothly and with little expenditure in terms of power.

Moreover, compared to conventional fans for respiration therapy appliances, the fan according to the invention can be operated at the same pressure with lower speeds of rotation. This in turn results in considerably less operating noise, such that therapy can be performed particularly quietly. It has also been found that the invention also provides considerably improved efficacy in the generation of the respiratory air flow. The invention therefore affords considerable advantages compared to other approaches for preventing exchange losses or exchange flows.

The winglet is preferably directed at least in the direction of a suction side of the blade element. The suction side here is in particular that side of the blade element facing away from a direction of rotation of the fan impeller. In particular, a pressure side is the side of the blade element facing toward the direction of rotation of the fan impeller. Such an arrangement of the winglets is particularly advantageous and for example, compared to a fan impeller without winglets, provides an increase in pressure and also an increase in efficacy.

It is also preferable that the winglet is directed in the direction of a pressure side and a suction side of the blade element. Such an embodiment is particularly advantageous for achieving an increase in pressure. It is also possible that the winglet is directed only in the direction of a pressure side of the blade element.

In a particularly advantageous embodiment, the winglet is arranged on the longitudinal side of the blade element in such a way that the winglet runs in the direction of a suction side to the same extent as in the direction of a pressure side of the blade element. In particular, the longitudinal side of the blade element runs centrally with respect to the winglet or is centered over the winglet. In particular, the winglet is arranged centrally on the blade element.

It is advantageous and preferable that the winglet has an extent of 1° to 20° of the circumference of the fan impeller. The winglet particularly preferably has an extent of 5° to 15° of the circumference of the fan impeller. The circumference of the fan impeller is in particular 360°. The extent of the winglet corresponds in particular to a sector of a circle. The extent of the winglet is in particular 10°+/−3° and preferably 10°+/−2° and particularly preferably 10°+/−1° of the circumference of the fan impeller. The extent of the winglet can also be 10°+/−0.5° or 10° of the circumference of the fan impeller. The extent of the winglets is preferably less than 21° and particularly preferably less than 20° of the circumference of the fan impeller. It is also possible that the winglet has an extent of up to 25° or more of the circumference of the fan impeller. Such dimensions of the winglets have proven particularly advantageous. These details relate in particular to the extent of the winglet over the entire length thereof or at the narrowest or broadest point thereof or at the center or end thereof.

In particular, an extent of the winglet increases from the radial interior of the fan impeller to the radial exterior of the fan impeller. It is possible that at least one other characteristic geometrical property of the winglet is adapted or modified from the radial interior to the radial exterior.

In an advantageous embodiment, the fan impeller can be equipped on at least one axial side, preferably on only one axial side, with at least one disk. The disk is preferably suitable and designed as a support disk for at least partially securing the blade elements. It is also possible that the disk is suitable and designed as a cover disk for at least partially covering the blade elements in terms of flow technology. It is a particular advantage of the invention that such a disk can be omitted at least on the side with the winglets. The winglets afford a particularly advantageous replacement for such a disk, since they permit a considerably lower weight and a quieter operation and also improved flow properties.

Provision can be made that the winglets are suitable and designed to replace disks on both axial sides of the fan impeller. It is possible that, despite the winglets, disks are provided on both axial sides of the fan impeller, for example for securing the blade elements or with a flow technology function.

The disk can be rigidly connected to the fan impeller. It is also possible that the disk is designed separate from the fan impeller or constitutes a component part separate from the fan impeller. In particular, the support disk is rigidly connected to the fan impeller and/or the cover disk is designed separate from the fan impeller.

The disk, in particular the cover disk and/or the support disk, has in particular a radially inward suction opening and/or outflow opening. It is possible that the blade elements protrude beyond the circumference of the cover disk. The cover disk is in particular not connected to the blade elements. In particular, the cover disk does not serve to secure the blade elements.

The disk, in particular the support disk and/or the cover disk, carries the blade elements. The support disk is in particular rigidly connected to the blade elements and is preferably designed in one piece with the blade elements, for example as part of a hub for passage of a drive shaft. The blade elements can protrude beyond the circumference of the support disk. The disk can be of a closed configuration.

The invention can also comprise a fan impeller without support disk and cover disk. The blade elements are then equipped in particular with winglets on both axial longitudinal sides. The blade elements can then be secured, for example, to a fan impeller wheel. For example, the fan impeller can then be designed as a star impeller or the like.

In an advantageous and preferred embodiment, the disk is arranged at least, in particular only, on that axial side of the fan impeller which lies opposite an axial side of the fan impeller equipped with the winglets. Thus, the winglets are arranged on one axial side of the fan impeller, and the disk is arranged on the opposite axial side. Such an arrangement of disk and winglets affords many advantages in terms of flow technology. The support disk is preferably arranged on that axial side of the fan impeller which lies opposite an axial side equipped with the winglets. In such an embodiment, the fan impeller can be designated as a support disk fan impeller without cover disk and with winglets. However, it is also possible that the cover disk is arranged in this way. In particular, the blade elements are arranged or secured on the disk with an axial longitudinal side that is not equipped with the winglets.

It is also possible for both axial sides of the fan impeller to be designed with winglets and without disks. In this case, the winglets provide an advantageous and lightweight replacement for both disks. Additionally or alternatively, at least one disk can also be arranged on the side with the winglets. Then, the circumference and/or a central opening of the disk are adapted in particular in terms of flow technology to the action of the winglets. For example, a disk is then provided having a circumference beyond which the blade elements protrude with their winglets.

The blade elements are preferably arranged within a circumference of the disk. In particular, the blade elements do not protrude beyond the circumference of the disk. In particular, the winglets also run within a circumference of the disk and/or do not protrude beyond the circumference of the disk. However, it is also possible that the blade elements and/or the winglets do protrude beyond the circumference of the disk.

In a particularly advantageous embodiment, the blade elements are at least partially and preferably completely straight. This permits particularly cost-effective and economical production of the fan impeller, for example by means of an injection molding method or the like. In particular, the blade elements are flat or planar. In particular, the surfaces of the blade elements are at right angles to the main plane of the fan impeller. For example, so-called 90° blades are provided. It is also possible to provide 90° blades+/−15°. It is also possible and preferable that the blade elements are curved. The blade elements can have a twisted or helical configuration over their longitudinal extent. The blade elements can also be bent, so as to be twisted about their longitudinal axis.

In particular, the blade elements of the fan impeller are all of identical configuration. However, it is also possible that the blade elements of the fan impeller are of different configurations. In particular, the blade elements are designed electively, for example alternately, with the aforementioned features.

The fan impeller is preferably produced or able to be produced by at least one injection molding method. Other suitable production methods can also be provided. In particular, the blade elements and/or the winglets and/or the hub are produced in this way. The fan impeller can be produced from plastic or metal or also from a composite material.

The fan impeller is in particular designed in one piece. In particular, the blade elements and the winglets and in particular also the hub are produced and connected to one another in one piece. It is also possible that the fan impeller is connected in one piece to the support disk and/or cover disk. The fan impeller can also be designed in several parts.

The fan impeller according to the invention is provided for a respiration therapy appliance preferably of the kind described above. The fan impeller comprises a plurality of blade elements. At least some of the blade elements are equipped with in each case at least one winglet running at least in part on at least one axial longitudinal side of the blade element. The fan impeller is preferably designed in the manner described above for the respiration therapy appliance according to the invention.

The invention set forth here can in particular be used in all suitable forms of fan impellers. The fan impeller can be propeller-like or designed as a propeller. The fan impeller can preferably be encased by at least one impeller housing. The housing has in particular at least one suction opening and at least one outflow opening. The fan impeller is in particular suitable and designed to suck air in axially and blow it out radially. Other arrangements can also be provided, for example air can be sucked in axially and blown out axially, or it can be sucked in radially and blown out radially. The fan impeller can be configured as a radial impeller or axial impeller.

The fan comprises in particular at least one drive device and for example at least one electric motor. The fan is in particular operatively connected to at least one control device and is controllable via the latter. For example, specific speeds of rotation for the fan impeller can be set. The fan comprises in particular at least one suction region and at least one output region. It is possible that the suction region and/or the output region is made available by at least one disk and/or at least one housing or the like.

According to the invention, a winglet is understood in particular as a planar structure which is arranged along a longitudinal side of the blade element and which is effective in terms of flow technology. In particular, the winglets of the individual blade elements are designed separately from one another and have in particular no connection to one another. In particular, the winglets are not interconnected in such a way as to result in a closed and for example disk-like structure. The winglets are in particular designed as axial flanges, or flanges on an axial longitudinal side of the blade elements. The flanges can be designed so as to form an L-shaped cross section of the blade element together with the winglet. The flanges can also be configured on both sides such that a T-shaped cross section is obtained. According to the invention, a winglet does not have to be arranged at an angle of 90° to the blade; the winglets can also be arranged at <90° or >90° with respect to the blade.

The flanges can be rounded. In particular, all the blade elements are equipped with in each case at least one winglet and in particular with only one winglet. It is also possible that every second or third or fourth blade element is equipped with at least one winglet. It is possible that a large majority of the blade elements are equipped with at least one winglet.

The winglet runs in particular along the entire axial longitudinal side of the blade element. It is possible that the winglet runs over at least half and in particular over at least 75% and preferably over at least 85% and particularly preferably over at least 90% of the length of the axal longitudinal side of the blade element. The winglet can also run over at least 95% or at least 98% of the length of the axial longitudinal side of the blade element. The winglet is in particular of a continuous configuration. The winglet can also have at least one interruption.

The winglet is in particular arranged on only one axial longitudinal side of the blade element. It is also possible that at least one winglet runs at least in part on both axial longitudinal sides of the blade element.

The winglets are in particular of identical configuration. The winglets are in particular of identical configuration if the winglets are arranged on identically configured blade elements. The blade elements can also have differently configured winglets.

### BRIEF DESCRIPTION OF THE DRAWINGS

Further advantages and features of the present invention will become clear from the description of the illustrative embodiments, which are explained below with reference to the accompanying drawings.

In the drawings:

FIG. 1 shows a purely schematic perspective view of a respiration therapy appliance according to the invention;

FIG. 2 shows a purely schematic perspective view of a fan impeller according to the invention;

FIG. 3 shows the fan impeller from FIG. 2 in a side view;

FIG. 4 shows the fan impeller from FIG. 2 in a plan view;

FIG. 5 shows a further fan impeller according to the invention in a perspective view;

FIG. 6 shows the fan impeller from FIG. 5 in a side view;

FIG. 7 shows the fan impeller from FIG. 5 in a plan view;

FIG. 8 shows a further fan impeller according to the invention in a perspective view;

FIG. 9 shows the fan impeller from FIG. 8 in a side view;

FIG. 10 shows the fan impeller from FIG. 8 in a plan view;

FIG. 11 shows a further fan impeller according to the invention in a plan view;

FIG. 12 shows a highly schematic graph with characteristic curves for fan impellers; and

FIG. 13 shows a further highly schematic graph with characteristic curves for fan impellers.

### DETAILED DESCRIPTION OF EXEMPLARY EMBODIMENTS OF THE INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

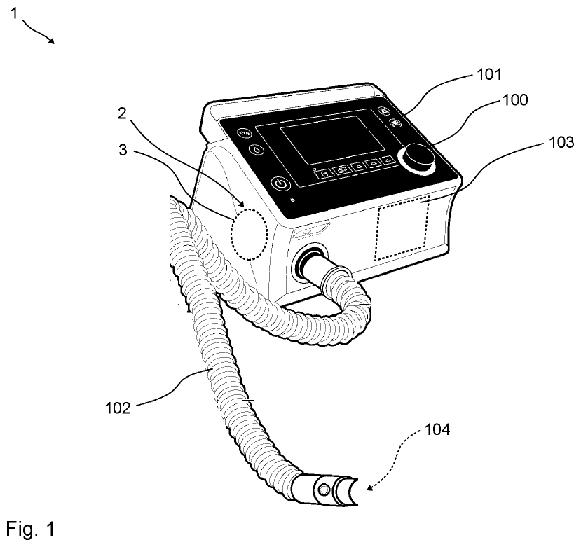

FIG. 1 shows a respiration therapy appliance 1 according to the invention, which is a respirator or a cough assistance appliance, for example. The appliance 1 is equipped with a fan 2 (accommodated in the appliance interior and therefore concealed here) which generates the respiratory air flow for the respiration therapy. The fan 2 is equipped with a fan impeller 3 according to the invention. The fan 2 has an electric drive for rotating the fan impeller 3.

The fan 2 is controlled by a control appliance 103 arranged and concealed in the appliance interior. For example, the control appliance 103 sets a defined speed of the fan impeller 3, according to the therapy requirements, or regulates the fan speed to a setpoint value.

The respiration therapy appliance 1 is here equipped with an operating device 100 and a display device 101. Some of the operations are performed via a touch-sensitive surface of the display device 101. The respiration therapy appliance 1 has an interface for coupling of a tube 102 for ventilation or cough assistance. The respiratory air flow generated by means of the fan 2 is delivered to the patient via the tube 102. A patient interface (not shown here) and for example a breathing mask can be attached to the tube 102.

FIGS. 2 to 4 show a fan impeller 3 according to the invention in different views. The fan impeller 3 is here equipped with a plurality of blade elements 4, with a disk 6 designed as a support disk 16, and with a hub 33 for connection to a drive shaft. The blade elements 4 are here secured to the support disk 16 and preferably also to the hub 33 and are for example connected to them in one piece. The fan impeller 3 can also be designed without disks 6 and in particular without the support disk 16, for example as a star impeller.

The preferred direction of rotation of the fan impeller 3 is illustrated here by an arrow. The blade elements 4 have a suction side 24 and a pressure side 34. The direction of rotation of the fan impeller 3 illustrated here results in the orientation of suction side 24 and pressure side 34 illustrated here.

In order to effectively and reliably suppress the exchange flow from the side space and also from the pressure side 34 to the suction side 24 in the direction of the side space or an axial side, the fan impeller 3 is equipped with winglets 5. Here, each blade element 4 is equipped with a winglet 5. The winglets 5 each run along an axial longitudinal side 14 of the blade element 4. In the embodiment of the invention shown here, the winglets 5 point in the direction of the suction side 24 of the respective blade element 4. For such a fan impeller 3, it was possible to observe an advantageous increase in pressure and also a considerable improvement in efficacy.

The fan impeller 3 shown here is, for example, integrated into the fan 2 such that air is sucked in axially and blown out radially. The suction side here lies at the axial side 23 with the winglets 5.

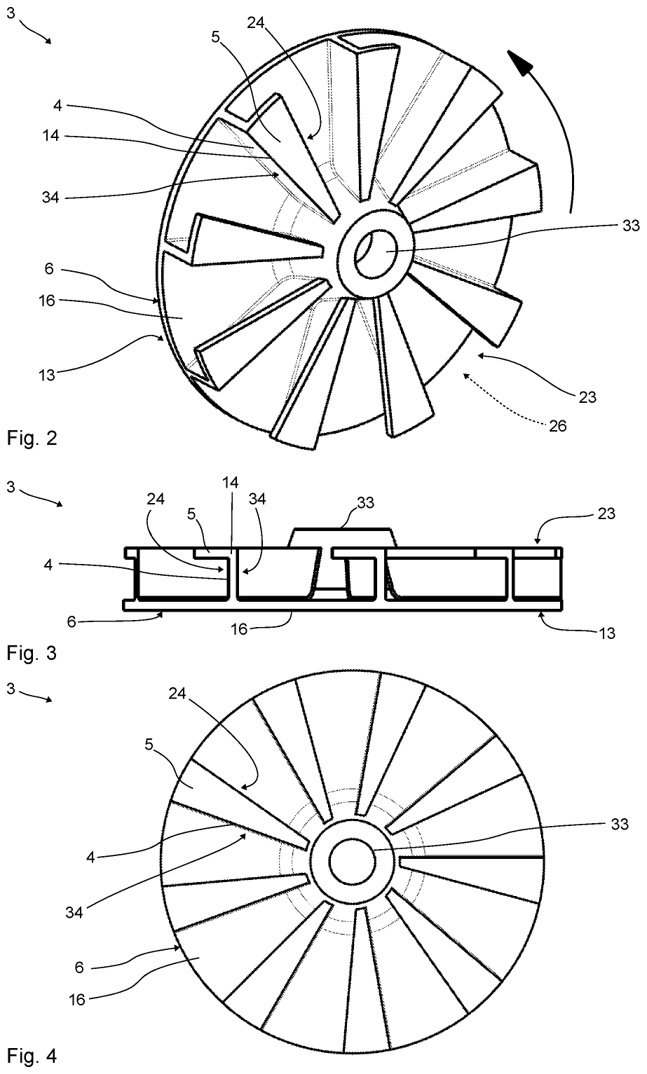

It will be seen from FIG. 3 that the material thickness chosen for the disk 6, the blade elements 4 and the winglets 5 is similar. According to the invention, provision is also made that the material thickness of the disk 6, of the blade elements 4 and of the winglets 5 is optimized according to the mechanical loads of the individual components mentioned, such that optimal stability is achieved, but at the same time with the lowest possible weight of the fan impeller 3. For example, the winglets 5 have a smaller material thickness than the disk 6. For example, the winglets 5 have a smaller material thickness than the blade elements 4, and the blade elements 4 have a smaller material thickness than the disk 6.

In the embodiment shown here, the fan impeller 3 is equipped with the disk 6 only on one axial side 13. The disk 6, designed as a support disk 16, is here arranged on that axial side 13 of the fan impeller 3 that lies opposite the axial side 23 of the fan impeller 3 equipped with the winglets 5.

In a development, a disk 6 designed as a cover disk 26 (not shown here) can also be provided. The cover disk 26 can be arranged on the axial side 23 additionally to the support disk 16 or can replace the support disk 16.

However, the axial side 23 with the winglets 5 is preferably not equipped with a disk 6, since the winglets 5 not only effectively suppress the exchange flow but at the same time also contribute considerably less weight than, for example, a cover disk 26.

In this way, the fan impeller 3 shown here provides particularly good flow properties and additionally has a particularly low inertia, such that the fan impeller 3 can be accelerated particularly smoothly. By means of the winglets 5, it is thus possible to dispense with a disk 6 on the axial side 23 without causing undesired exchange flows and therefore unfavorable losses. With the fan impeller 3 according to the invention, the speed of rotation can also be adapted in a much less complicated way and more rapidly. A further advantage is that, by virtue of the winglets 5, more pressure can be generated than is possible in a fan impeller without winglets, and therefore the fan impeller 3 can be operated at a lower speed to achieve the same operating point. This results in a much quieter operation of the respiration therapy appliance 1, such that the therapy is felt to be more comfortable and more pleasant.

The fan impeller 3 shown here has straight blade elements 4. However, other configurations are also possible, for example with curved blade elements 4. Such a configuration can be produced particularly advantageously by injection molding. The fan impeller 3 shown here is produced in one piece.

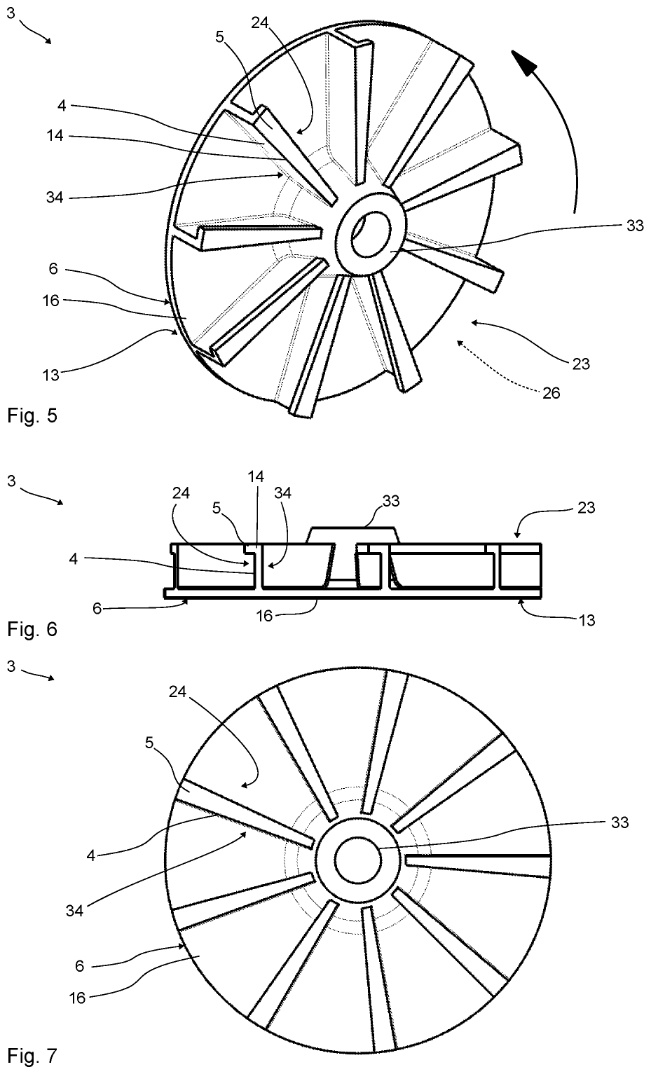

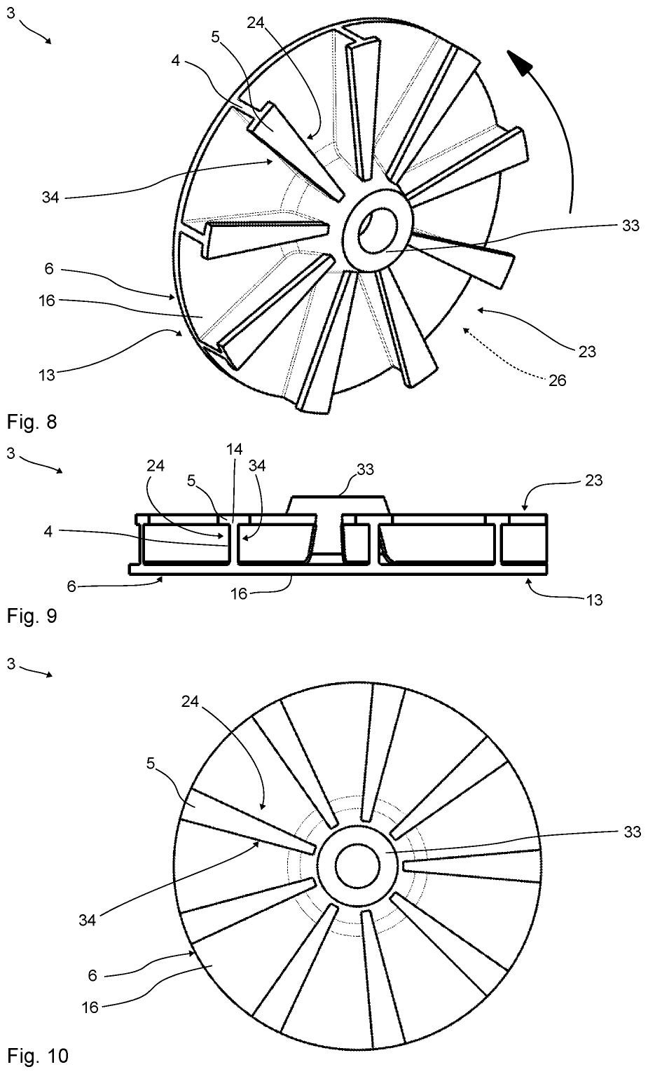

FIGS. 5 to 7 show a fan impeller 3 according to the invention that has shorter winglets 5 than the impeller wheel 3 described above.

FIGS. 8 to 10 show a fan impeller 3 according to the invention that is equipped with winglets 5 on both sides of the blade elements 4. The winglets 5 run both in the direction of the pressure side 34 and also in the direction of the suction side 24 of the blade elements 4. The winglets 5 here are centered on the longitudinal sides 14 of the blade elements 4, such that the winglets 5 run to the same extent in the direction of the suction side 24 and in the direction of the pressure side 34. For such a fan impeller 3, a particularly advantageous pressure increase could be observed.

If the winglets 5 are arranged on both sides 24, 34 of the blade elements 4, particularly advantageous flow properties are achieved if they are made suitably short, for example as shown here.

FIG. 11 shows a fan impeller 3 in which the winglets 5 have an extent of in each case 10° of the circumference of the fan impeller 3. For winglets 5 of such dimension, particularly good flow properties could be demonstrated. Advantageous flow properties could also be observed for dimensions of between 5° and 15° of the circumference of the fan impeller 3.

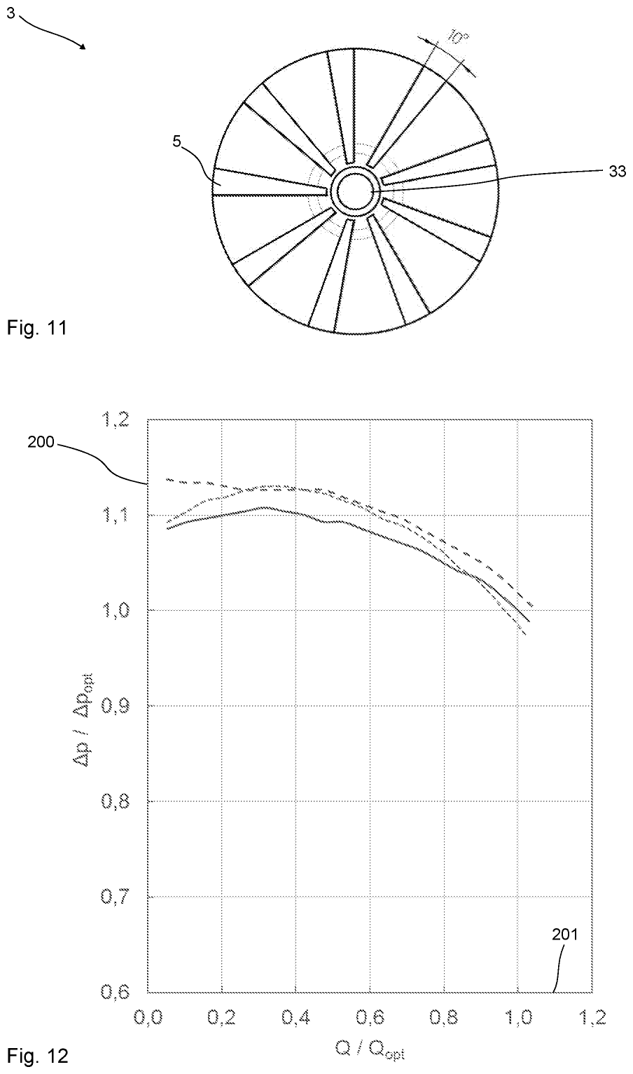

FIG. 12 shows three different pressure curves in diagrammatic form, in which the pressure has been plotted against the volumetric flow. The axes are standardized here to an optimal operating point of the configuration without winglets 5. The solid curve corresponds to a fan impeller 3 without winglets 5. The pressure curve with the long-dashed line was found in a fan impeller 3 with winglets 5 that run in the direction of the suction side 24 of the blade elements 4. The pressure curve with the short-dashed lines was found in a fan impeller 3 whose winglets 5 run in the direction of the pressure side 34 of the blade elements 4. The winglets have an extent of 10° of the circumference of the fan impeller 3.

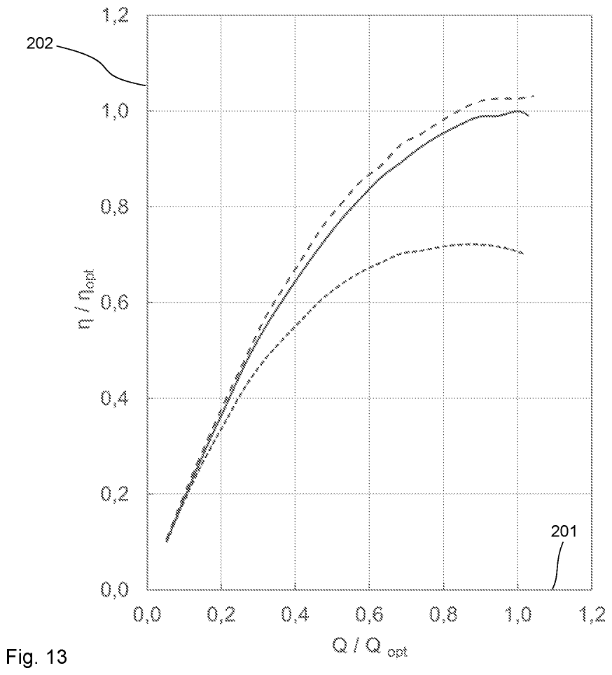

FIG. 13 shows efficacy curves in which the degree of efficacy has been plotted against the volumetric flow. The axes here are standardized to an optimal operating point of the configuration without winglets 5. The solid efficacy curve corresponds to a fan impeller 3 without winglets 5. The efficacy curve with the long-dashed line was found in a fan impeller 3 with winglets 5 that run in the direction of the suction side 24 of the blade elements 4. The efficacy curve with the short-dashed lines was found in a fan impeller 3 whose winglets 5 run in the direction of the pressure side 34 of the blade elements 4. The winglets have an extent of 10° of the circumference of the fan impeller 3.

### LIST OF REFERENCE NUMERALS

1 respiration therapy appliance

2 fan

3 fan impeller

4 blade element

5 winglet

6 disk

13 side

14 longitudinal side

16 support disk

23 side

24 suction side

26 cover disk

33 hub

34 pressure side

100 operating device

101 display device

102 tube

103 control device

104 patient interface

200 pressure

201 volumetric flow

202 efficacy

What is claimed is:

1. A respiration therapy appliance, wherein the appliance comprises at least one fan for generating a respiratory air flow for carrying out respiration therapy, the at least one fan comprising at least one rotatable fan impeller comprising a plurality of blade elements, and at least some of the plurality of blade elements being equipped with in each case at least one winglet running at least in part on at least one axial longitudinal side of the blade element.

2. The respiration therapy appliance of claim 1, wherein the winglet is directed at least in a direction of a suction side of the blade element.

3. The respiration therapy appliance of claim 1, wherein the winglet is directed in a direction of a pressure side and a suction side of the blade element.

4. The respiration therapy appliance of claim 1, wherein the winglet is arranged on the axial longitudinal side of the blade element in such a way that the winglet runs in a direction of a suction side to the same extent as in the direction of a pressure side of the blade element.

5. The respiration therapy appliance of claim 1, wherein the winglet has an extent of 1° to 20° of a circumference of the fan impeller.

6. The respiration therapy appliance of claim 5, wherein the winglet has an extent of 5° to 15° of the circumference of the fan impeller.

7. The respiration therapy appliance of claim 1, wherein the winglets run in a direction of a pressure side of the blade elements and have an extent of 2°-20° of a circumference of the fan impeller.

8. The respiration therapy appliance of claim 1, wherein an extent of the winglet increases from a radial interior of the fan impeller to a radial exterior of the fan impeller.

9. The respiration therapy appliance of claim 1, wherein the winglets comprise curved blade elements.

10. The respiration therapy appliance of claim 1, wherein the fan impeller is equipped on only one axial side with at least one disk which is configured as a support disk for at least partially securing the blade elements and/or wherein is configured as a cover disk for at least partially covering the blade elements in terms of flow technology.

11. The respiration therapy appliance of claim 10, wherein the at least one disk is arranged only on that axial side of the fan impeller which lies opposite an axial side of the fan impeller equipped with the winglets.

12. The respiration therapy appliance of claim 11, wherein the at least one disk is configured as a support disk.

13. The respiration therapy appliance of claim 10, wherein the blade elements are arranged within a circumference of the at least one disk and/or do not protrude beyond the circumference of the at least one disk.

14. The respiration therapy appliance of claim 1, wherein the blade elements are at least in part straight or curved.

15. The respiration therapy appliance of claim 1, wherein the fan impeller is produced in one piece with the winglets by an injection molding method.

16. The respiration therapy appliance of claim 10, wherein a material thickness of the at least one disk, the blade elements and the winglets is optimized according to mechanical loads, such that optimum stability is achieved and, at the same time, the fan impeller as a whole has the lowest possible weight.

17. The respiration therapy appliance of claim 10, wherein the winglets have a smaller material thickness than the at least one disk.

18. The respiration therapy appliance of claim 1, wherein the winglets have a smaller material thickness than the blade elements.

19. A fan impeller for a respiration therapy appliance, wherein the impeller comprises a plurality of blade elements, at least some of the plurality of blade elements being equipped with in each case at least one winglet running at least in part on at least one axial longitudinal side of a blade element.

20. A respiration therapy appliance, wherein the appliance comprises at least one fan for generating a respiratory air flow for carrying out respiration therapy, the fan comprising at least one rotatable fan impeller comprising a plurality of blade elements, wherein at least some of the plurality of blade elements are equipped with in each case at least one winglet running at least in part on at least one axial longitudinal side of the blade element, wherein the winglets are directed at least in part in a direction of a pressure side of the blade elements and have an extent of 2° to 20° of a circumference of the fan impeller, wherein the fan impeller is equipped on only one axial side with at least one disk which is configured as a support disk for at least partially securing the blade elements and/or is configured as a cover disk for at least partially covering the blade elements in terms of flow technology, wherein the at least one disk is arranged only on that axial side of the fan impeller which lies opposite an axial side of the fan impeller equipped with the winglets, and wherein the blade elements are arranged within a circumference of the at least one disk and/or do not protrude beyond the circumference of the at least one disk.
